(19) United States
(12) Patent Application Publication
(10) Pub. No.: US 2008/0287881 A1
(43) Pub. Date: Nov. 20, 2008

(54) Syringe for use with nuclear medicines

(76) Inventor: Bruce Leigh Kiehne, Springwood (AU)

Correspondence Address: HOFFMAN, WASSON & GITLER, P.C.;Suite 522, 2461 South Clark Street, Arlington, VA, 22202, US

(21) Appl. No.: 11/798,363

(22) Filed: May 14, 2007

(51) Int. Cl. A61M 5/32

(52) U.S. Cl. 604/195

(57) ABSTRACT

A no leak syringe has:
a syringe body having a front portion and an open rear end portion,
a plunger having a front and a rear and which is slideable along the barrel from a retracted position where the front of the plunger is in the rear portion of the barrel to a extended position where the plunger has been slid along the barrel towards the front of the barrel,
a pierceable self healing seal extending over the front of the plunger and through which the needle can pierce when the needle is retracted, the seal being substantially closed after the needle has passed through the seal,
a needle holder which is positioned in the front of the barrel,
a needle attached to the needle holder,
a triggering mechanism to enable the needle to be retracted into the syringe body and/or the plunger body,
a plunger lock mechanism to lock the plunger against retraction when the needle has been retracted,
a thumb pad on the rear of the plunger, and
a seal on the thumb pad that seals against the open rear end portion of the syringe body to prevent liquid from leaking from the rear end portion of the barrel when the needle has been retracted.

### FIELD OF THE INVENTION

This invention is directed to a medical safety syringe and typically a syringe that has a shoot back type needle retraction mechanism, and which has a special design to create a sealed chamber after retraction of the needle, thereby making the syringe a “no leak” syringe and allowing the syringe to be used with medicines which are hazardous such as radioactive ( nuclear) medicines or other hazardous substances.

### BACKGROUND OF THE INVENTION

In the medical field, needle stick is a particular occupational hazard and therefore many devices have been manufactured to reduce the needle stick hazard. Needlestick from syringe needles is a particular hazard for medical practitioners. It is also advantageous to ensure that a medical syringe cannot be reused. This prevents contamination. For this reason, there are many syringes having mechanisms or means to restrict the syringe to a single use syringe.

One known mechanism is a “shoot back” mechanism developed by the present applicant, and which is the subject of previous patents. This mechanism comprises a needle holder in the front part of the syringe barrel and to which the needle is attached. A spring is placed under compression about the needle holder. The needle holder is held in place against the compression of the spring in the front of the syringe. When the plunger is pushed through the syringe barrel and towards the front of the barrel, the front of the plunger dislodges or otherwise actuates part of the needle holder which releases the spring compression and causes the remainder of the needle holder+the needle to shoot back into the safety of the plunger body. In this particular design, the needle holder has a certain thickness and therefore the plunger triggers the shoot back mechanism before the plunger is able to travel to the front most part of the barrel.

There are various other mechanisms to retract the needle. These may include different type of spring arrangements, different type of needle holder designs, different type of plunger designs, springs that “stretch” as the plunger is moved forwardly, designs where the plunger locks to the needle holder and retraction of the plunger retracts the needle holder and the like.

It is sometimes necessary to inject a radioactive medicine (a nuclear medicine) or to load the syringe with other types of hazardous materials. Great care must be taken that the hazardous materials do not come in contact with the medical practitioner. Therefore, it would be advantageous if the syringe could have a retractable needle and that the design of the retraction mechanism and the design of the syringe is such that when the needle is retracted (typically into hollow plunger body), the syringe is substantially sealed such that any residual radioactive material or hazardous residual material on the needle cannot leak from the syringe.

It is found that many needle retraction mechanisms leave a rather large opening when the needle is in the retracted position which means that any hazardous material that is still in the needle can leak from the syringe. As an example, a known type of retraction mechanism has a hollow plunger and the front of the plunger contains some form of shatter plate or dislodgeable member and when the needle is retracted the needle shatters the shatter plate or pushes back the dislodgeable member. While this safely retracts the needle into the hollow plunger body, it results in the front of the plunger remaining wide-open and this means that potentially hazardous materials can leak from the plunger and into the syringe and out the now open front end of the syringe (where the needle was).

Conversely, the design of the retraction mechanism (and particularly the design of the front of the plunger) in our earlier patents did not leave a large opening in the front of the plunger after the needle had been retracted into the plunger body. Specifically, the front of our plunger contained a sealing member which was relatively thin, and when the needle retraction mechanism was triggered, the needle could “punch through” the sealing member and into the plunger body. The sealing member was made of resilient material and was “self-healing” which meant that after the needle had been retracted into the plunger body, the front of the plunger was still sealed because of the self-healing nature of the plunger seal. Therefore, any potentially hazardous liquid still remaining in or on the needle could not leak through the front of the plunger. When the needle and needle holder was retracted into the plunger cavity, the needle holder would be “locked” in the rear of the plunger cavity by engaging with small catches that were positioned in the rear of the plunger body, and the catches were manufactured by forming small openings in the wall of the plunger body. Thus, it was still possible for potentially hazardous material to leak through these small openings.

The present invention is directed to an improvement over our earlier syringe designs and particularly improvement(s) which now makes it very difficult, if not impossible, for residual liquid to leak from either the front of the syringe or the rear of the syringe once the needle has been retracted. This would be so irrespective of how the syringe was held. Thus, the present invention is directed to a syringe design which prevents leaking from the syringe after the needle has been retracted even if the syringe is held vertically, at an angle, horizontally and the like. There would also be an advantage to provide design features that could prevent the plunger from being retracted after the needle retraction mechanism has been triggered.

It is believed that there is no other single use syringe in the marketplace which can be used for nuclear medicines and which has an automatically retracting needle and when the needle has been retracted, the syringe is completely closed off and safe to dispose.

### BRIEF SUMMARY OF THE INVENTION

It is an object of the invention to provide a syringe or similar device that may overcome at least some of the above-mentioned disadvantages or provide a useful or commercial choice.

In one form, the invention resides in a syringe comprising:

a syringe body having a front portion and an open rear end portion,

a plunger having a front and a rear and which is slideable along the syringe body from a retracted position where the front of the plunger is in the rear portion of the syringe body to a extended position where the plunger has moved along the syringe body towards the front of the syringe body,

a pierceable self healing seal extending over the front of the plunger and through which the needle can pierce when the needle is retracted, the seal being substantially closed after the needle has passed through the seal,

a needle holder which is positioned in the front of the syringe body,

a needle attached to the needle holder,

a triggering mechanism to enable the needle to be retracted into the syringe body and/or the plunger body,

a plunger lock mechanism to lock the plunger against retraction when the needle has been retracted,

a thumb pad on the rear of the plunger, and

a seal on the thumb pad that seals against the open rear end portion of the syringe body to prevent liquid from leaking from the rear end portion of the syringe body when the needle has been retracted.

FIGS. 9-12 illustrate the self-healing seal, the plunger lock mechanism, the thumb pad and the seal on the thumb pad.

The needle holder may comprise a design described in one or more of our earlier patent applications but may also comprise a design described in other patent applications or in other commercial products or published products. Usually, the needle holder will contain some part which can contain the needle and this part is usually a central part or inner part of the needle holder. The needle holder or part of the needle holder will typically be held in some releasable manner in a forward part of the syringe body and can be “triggered” or otherwise manipulated to cause retraction of the needle. In our earlier patent applications, the needle holder is held in a front part of the syringe body by a step or something similar. In other designs, the needle holder is held in the front part of the syringe body by other means including friction, a shatter ring and the like.

Part of the needle holder is typically biased by a spring or something similar to shoot back into the plunger body/syringe body but is prevented from doing so by being held in some manner and this may comprise a frangible portion and the like.

The triggering action to release part of the needle holder (containing the needle) for retraction usually occurs by part of the plunger contacting part of the needle holder when the plunger has been pushed in the fully forward position.

These mechanisms of retraction are described in some of our earlier patent applications and in other publications.

The needle holder may be attached inside and in the front area of the syringe body. A spring may be provided in the front of the syringe body and that is biased to shoot part of the needle holder through the pierceable seal in the front face of the plunger and into the plunger. The needle may have an inner part and an outer part, the inner part containing the needle, the outer part engaging with a wall of the longitudinal passageway and initially in a rear portion of the passageway, a frangible portion between the inner part and the outer part. The outer part may contain a raised portion ( which may be called a “bump”) directed towards the seal of the plunger, the raised portion extending only partway about the inner portion, the raised portion functioning to provide an initial contact area of the plunger with the outer part of the needle holder, thereby reducing the force required to break the frangible portion thereby triggering the shoot back mechanism.

The raised portion (bump) may contain an uppermost portion adapted for contact with the plunger, and a shoulder portion adjacent the uppermost portion and which functions to reduce dead space between the plunger head and the needle retracting mechanism. The shoulder portion may be called a “chamfer”. The shoulder portion can function to fill the otherwise dead space and therefore reduce the volume of dead space which may otherwise be present between the plunger head and the shoot back mechanism. It is envisaged that the shoulder portion will be positioned adjacent each side of the uppermost portion and it is envisaged that the shoulder portion and the uppermost portion will comprise a unitary “raised portion” on the outer part. The shoulder portion may comprise a “chamfer” trailing down from the uppermost portion to the surface of the outer part. The shoulder portion may have a relatively smooth incline, or may have an irregular shape or any other type of shaped or configuration that can reduce the dead space while not undesirably affecting the shoot back mechanism, and especially the reduced force shoot back mechanism.

The plunger can have a conventional flat front face on the front end and can be fitted with a pierceable self healing seal to minimise any interference with the needle holder shooting back into the plunger body and through or past the seal. Thus, there is no requirement for a relatively bulky stopper or plug to be fitted to the front of the plunger. There is also no need for the front of the plunger to contain a cutting face.

The triggering mechanism may use a push and break movement where the outer part of the needle holder is pushed forwardly by the plunger along the longitudinal passageway while the inner part remains stationary, and this movement breaks the frangible portion that holds the inner part to the outer part. This is considered advantageous over a cutting action or any other type of triggering action. However, a main advantage is that the force required to break the frangible portion and therefore trigger the shoot back mechanism is reduced due to the design of the outer part and particularly due to the raised portion (bump) that faces the front of the plunger and which provides the initial point of contact between the plunger and the outer part. The design also can reduce dead space, as well as enabling the plunger to be fitted with a pierceable seal as opposed to the complex design of the plunger that has a forwardly projecting part and which may be difficult to contain a pierceable seal.

It is preferred that the seal that extends over the front face of the plunger contains a central portion that extends over the otherwise hollow front of the plunger, and an edge portion that extends over the edge of the plunger. The central portion can be designed to be more easily pierced or broken, for instance by making the wall thickness less. The edge portion may contain a thicker area of seal and this area can be compressed or squashed as it contacts the raised portion of the outer part. The advantage of this is that the front of the plunger can be pushed quite close to the needle holder (thereby reducing dead space) before the force is sufficient to trigger the shoot back mechanism.

The syringe may be of any suitable shape and size. It is envisaged that the syringe will vary between a 1 mil syringe up to a 50 mil syringe or even more. The syringe may be made of any suitable material such as plastic, glass or even metal. It is preferred that the syringe is made of plastic.

The syringe has a syringe body. The body will typically be cylindrical in design as is conventional, although if necessary, the body may have other shapes such as rectangular in cross-section, oval in cross-section and the like. The length of the syringe body will vary depending on the size of the syringe and will typically be between 30-200 millimetres although this can vary. The diameter of the syringe body may also vary and will typically be between 5-20 millimetres although this can vary. The syringe body will typically have a wall thickness of between 0.3-4 mm although this can vary. The syringe body may be provided with grip enhancing features or positioning enhancing features such as outwardly extending tabs or flanges, exterior ribs and the like.

The syringe body may have a front portion from which the puncture needle will project. The front portion will typically have a front opening through which the needle can extend. The front portion will contain a longitudinal passageway which will typically be designed such that the plunger can pass along or at least partially along this passageway, and the passageway forms part of the shoot back mechanism which will be described in greater detail below. The passageway will typically have a front portion which is more towards the front opening through which the needle extends, and a rear portion. The size and shape of this passageway can vary, but it is envisaged that this passageway may comprise a continuation of the internal bore of the syringe body. The front passageway may have a length of between 5-30 mm depending on the size of the syringe.

The syringe will have a plunger. The length of the plunger will vary depending on the size of the syringe and the plunger will typically have a length of between 30-200 mm although this can vary. The plunger is designed to slide in the syringe body and therefore the plunger will have a diameter or cross-section which enables it to do so. The plunger will typically be substantially or entirely hollow such that the contaminated needle can be shot back into the hollow interior of the plunger. However, it may also be possible to have the forward part of the plunger hollow (sufficiently to at least partially hold the needle) and the rear part solid or of different design. It is also envisaged that the plunger need not be hollow and may comprise an X type cross-section, or other cross-section such that the contaminated needle can pass into the passageway which is defined between the cross-section and the internal wall of the syringe. Other configurations of the plunger are envisaged providing that space is provided by the inside the plunger or between the plunger and the syringe body to accommodate the contaminated needle after the shoot back mechanism is triggered. However, it is considered useful that the plunger comprises a hollow cylindrical member.

The plunger has a front end formed with a front face which is the part of the plunger that extends into the syringe body and is pushed towards the front of the syringe. If the plunger is cylindrical, the front face will also be substantially cylindrical.

A seal is provided over the front face of the plunger. The seal is of the type that can be pierced such that the contaminated needle can pierce through or pass by the seal when the shoot back mechanism is triggered. Various types of seals are envisaged including rubber seals, plastic seals, Santoprene™ seals, elastic seals, laminated seals and the like. The seal will typically extend entirely over the front face of the plunger although depending on the size of the needle and the needle body, the seal may pass only over a central portion or other portion of the plunger. It is however preferred that the seal extends entirely over the front face of the plunger. If desired, the seal may have a central portion that extends over the central portion of the plunger and which can be of a different design (typically thinner) to enable it to be pierced more easily, and an edge portion that extends over the edge of the plunger and which may be thicker and more easily compressed or squashed, the reason for which will be described in greater detail below. The seal may also extend over the sides of the plunger to provide a good sealing effect and to also assist in sealing the plunger against the inside wall of the syringe body. Alternatively, the seal that extends over the front face of the plunger may be different and separate to the seal that extends over the side wall of the plunger, and which functions to prevent liquid from flowing behind the plunger seal.

The seal that extends over the front face of the plunger is designed to be self-healing or self-closing once the needle has pierced through the seal. There are various elastomeric and other types of products that can provide this self-healing property. Thus, the seal is pierced to allow the needle+part of the needle holder+part or all of the spring to pass through the seal and thereafter the seal closes (heals) to prevent any potentially hazardous liquid from passing through the seal.

The syringe contains a needle holder that holds the needle. The needle holder will typically be attached inside and in the front area of the syringe body. The needle holder may comprise an inner part and an outer part which are attached to each other by a frangible portion. Thus, it is not preferred that the inner part and the outer part are engaged frictionally; instead, the inner part and the outer part are bonded or joined together via the frangible portion. This provides a more reliable action and minimises accidental release of the needle holder.

The inner part may comprise a front portion and a rear portion and an intermediate body portion. The front portion may extend through the opening in the front of the syringe body. The intermediate body portion may comprise an elongate substantially cylindrical portion containing an internal passageway which communicates with the puncture needle. The rear portion may extend towards the plunger and may comprise a profile or have a projection or have any other configuration to assist in the piercing or breaking of the plunger seal. Typically, the rear portion will have an “arrowhead” type portion or configuration.

A spring may be provided to bias the needle holder towards the retraction or shoot back position. The spring may comprise a helical spring and the helical spring may extend about the intermediate body portion of the needle holder.

The outer part of the needle holder may comprise the part that holds the needle holder in position in the front part of the syringe body and against the bias of the spring. The outer part may comprise an annular member. The annular member may have an outer face that engages with the inner wall of the syringe body. The engagement may comprise frictional engagement, but may also comprise abutment of part of the outer face with a shoulder, recess, or other configuration of the syringe body.

The outer part of the needle holder will typically be positioned in a rear portion of the passageway that is in the front of the syringe body. Thus, the outer part will typically be able to be pushed along the passageway from the rear portion of the passageway towards the front portion of the passageway. This distance may be between 1 mm up to 20 mm depending on the size of the syringe. Movement of the outer part in this manner relative to the inner part will cause the frangible portion to break thereby releasing the inner part from the outer part and triggering the shoot back mechanism.

Pushing of the plunger to the front of the syringe may cause the plunger to engage with the outer part and to push the outer part along the passageway thereby breaking the frangible portion and triggering the shoot back mechanism.

The outer part will typically contain at least one raised portion/zone/area which is directed towards the front of the plunger, that is which is pointing inwardly into the syringe body. The raised portion etc may be formed integrally with the outer part or may be formed separately and attached thereto, or relative thereto. The raised portion may comprise a bump, a rib, a tooth, a “button”, or any other type of configuration which will serve the purpose. It is preferred that the raised portion extends only partially about the outer part, or only partially about the inside of the syringe. The arrangement will typically be such that as the plunger is pushed forwardly, the plunger will contact the raised portion first before the rest of the outer part is contacted by the plunger. This provides several benefits including a reduction in the force required to trigger the shoot back mechanism. For instance, the arrangement can concentrate the initial force of the plunger into a smaller area (typically on the outer part of the needle holder) to cause this part to move forwardly to break the frangible portion in this area. It is found the once the frangible portion is at least partially broken, less force is required to entirely rake the outer part from the inner part.

It is preferred that the edge portion of the seal on the plunger is thickened and is also preferred that this portion is relatively compressible. The reason for this is that the edge portion will be the first point of contact with the raised portion of the outer part, and as this contact occurs, the edge portion is compressed before the frangible portion is broken. This compression enables the front of the plunger to be pushed more closely against the needle holder thereby reducing dead space prior to triggering the shoot back mechanism.

The shoot back mechanism has similarities to that described in our earlier PCT application PCT/AU01/00183 which is incorporated here in by cross-reference.

If desired, the syringe may be provided with position indicators to allow a medical practitioner to more clearly determine the “status” of the syringe. To explain, one disadvantage with conventional needle retraction syringes is that it is sometimes difficult to clearly see the “status” of the syringe. One “status” of the syringe is when the plunger has been fully pulled back (retracted) and medicine has been introduced into the syringe barrel. Typically, this status is quite easily observed as the plunger is fully retracted and the syringe barrel is usually clear or translucent and the (usually) black plunger seal is clearly visible in the rear part of the syringe.

As the plunger is pushed forwardly, medicine is ejected through the needle, and the plunger moves towards the “triggering” or retraction mechanism. Sometimes, there is an advantage in slowing the plunger movement just before the triggering. However, unless the plunger is pushed fully against the triggering mechanism, there may be incomplete injection of the medicine into a patient (that is, some medicine may still be in the syringe) and the triggering mechanism will not function.

For the smaller syringes (e.g. five mill syringes or even smaller), the position of the plunger, when it gets close to the triggering mechanism, is often quite difficult to see. Some practitioners may push the plunger too quickly against the triggering mechanism causing the triggering mechanism to activate very quickly and this may cause alarm to a patient (or even the practitioner). Other times, the practitioner may take too long to slowly push the plunger against the triggering mechanism which may also cause alarm and distress to the patient.

Therefore, there would be an advantage to provide a syringe with some form of means or mechanism to clearly indicate the “status” of the syringe.

Another disadvantage with some safety syringes is that it is sometimes not entirely clear when the syringe has been “made safe”. To explain, some triggering mechanisms may require the plunger to be pushed past the initial triggering position. There may also be some safety syringes that lock the plunger in the fully forward position such that the plunger cannot easily be pulled out of the barrel. The position of the plunger to lock the plunger or to otherwise make the syringe safe may not be very easily observed by practitioners. Thus, the practitioner may place too much force on the plunger, or not enough force. Therefore, there would be an advantage if it were possible to provide a syringe, or other medical device that would benefit from the advantage, with some form of means or mechanism to clearly indicate the “made safe” position of the plunger in the syringe body.

The syringe may have a first visual indicator positioned at or adjacent the triggering position, and a second visual indicator positioned forwardly of the first visual indicator, the barrel being at least partially clear between the first visual indicator and the second visual indicator such that the plunger seal can be viewed therebetween when the plunger is pushed into that position, the construction and arrangement being that when the plunger seal is adjacent the first visual indicator, there is a visual indication that the triggering action is about to begin, and when the plunger seal can be viewed through the barrel between the first visual indicator and the second visual indicator, the plunger is in the locked position against retraction.

In this manner, a practitioner can very easily see when the plunger is about to trigger the retraction mechanism and this is usually the position where all the medicine in the syringe has been injected into the patient. Further forward movement of the plunger will position the plunger seal (which is usually black and therefore easy to see) between the first and second visual indicator and when the plunger is in this position, the plunger is locked and the syringe is “made safe”. Thus, a practitioner does not need to do anything more then view the plunger seal between the visual indicators to be certain that the plunger is now locked and there is no need for the practitioner to “fiddle” with the syringe just to make sure that the plunger is locked. Also, the practitioner can very easily see when the triggering mechanism is about to operate and therefore when the fluid in the syringe has been expelled.

Various types of visual indicators are envisaged. In a simple form, the, or each visual indicator may comprise a coloured band extending about the syringe barrel. The coloured band may be positioned inside the barrel or outside the barrel or be part of the barrel wall. The coloured band may comprise a paint, a coating, an ink or something similar, a separate thin member which can be glued or otherwise applied to or attached to the barrel and the like. It is not considered that any particular limitation should be placed on the type of coloured band.

The band may have any suitable contrasting colour and may comprise black, blue, red, green and the like. Each visual indicator may comprise a different colour. Each visual indicator may comprise a combination of colours. The band may comprise fluorescent material or luminescent material. The width of each visual indicator may vary, and this may depend inter alia on the size of the syringe. It is envisaged that, in respect of a small syringe (typically 1-3-mill), the width of the visual indicator may be between 1-3 mm. This can vary to suit.

It is preferred that the first and the second visual indicator are quite clearly separated such that the plunger seal can be clearly viewed between the indicators. The spacing between the first and second visual indicator can vary inter alia on the size of the syringe but it is envisaged that in respect of a small syringe, the spacing may be between 2-8 mm. This can of course vary to suit.

The positioning of the first and second indicator will depend on the type of needle holder, the type of syringe, the type of triggering mechanism and the type of piston lock mechanism that is used. One of the indicators should be positioned such that when the piston seal is at or close to the indicator, this indicates that the triggering mechanism is about to activate and/or that the contents of the syringe has been expelled fully. The other of the indicators should be positioned such that when the piston is locked against retraction, the seal is positioned between the visual indicator.

It is envisaged that a syringe will be provided having at least one indicator which may identify the point of triggering and one identification means which may identify the correct positioning of the plunger to engage the plunger lock mechanism. It is envisaged that a single indicator may be provided. For instance, when the plunger seal is to one side of the single indicator, this may be at the point of triggering and when the plunger seal is on the other side of the single indicator this may confirm that the plunger has been locked.

While it is envisaged that the indicators will comprise a band extending about the barrel (the advantage being that the indicators can function no matter how the syringe is held), there may be circumstances where the indicator extends only partially about the barrel. It is also envisaged that the indicators may comprise a series of spaced apart indicators which may extend about the barrel as opposed to a continuous band. It is also envisaged that the indicators may comprise any other suitable shape (that is not necessarily a band shape).

The indicators may also comprise indicia as opposed to a coloured band. For instance, the indicator may comprise an arrow or something similar to indicate the triggering point or the plunger locking point when the plunger seal is adjacent the arrow. The indicators may comprise other types of shapes.

While it is considered expedient that the plunger seal is used as an identifier when positioned next to a visual indicator, it is envisaged that there may be circumstances where other parts of the plunger may be used. As an example, the plunger may contain a band of contrasting colour or a visual indicator as described above, and this may be provided somewhere on the plunger body. A corresponding band or other type of identifier may be placed at a strategic location on the syringe body and proximal positioning may indicate that the plunger is locked or the triggering mechanism is about to activate etc.

The visual indicators may be coded ( e.g. color coded) to distinguish between different size (gauge) needles. This one color could be used for a 22 gauge needle and another colour could be used for a 23 gauge needle etc.

The syringe is provided with a plunger lock mechanism to prevent the plunger from being retracted when the needle has been triggered into the retracted position. This is usually the position where the plunger is pushed hard up against the front of the syringe barrel. The plunger lock mechanism may comprise a projection such as a locking collar extending from the plunger body and which engages with some part of the syringe body when the plunger is in the forward position thereby preventing retraction of the plunger. The projection may be positioned adjacent a rear portion of the plunger body. If desired, a number of projections may be provided although it is considered suitable that the projection comprises a circumferential extending collar or something similar. The collar, in a particular embodiment of the invention, is illustrated in FIG. 12 as reference numeral 72. The collar (72) may comprise a tapered edge (see FIG. 12). The function of the tapered edge is to enable the projection to initially push past the “engagement part” of the syringe body such that the plunger can be pushed to the front of the syringe body but is unable to be retracted subsequently.

The engagement part of the syringe body may comprise an inwardly extending abutment or shoulder or something similar. An example of this is illustrated in FIG. 12. It is considered suitable that the engagement part comprises a circumferential abutment although there may be circumstances where the engagement part may comprise a non continuous abutment or several other types of abutments. The arrangement is suitably such that the plunger can initially be pushed to the front of the syringe body (to expel the medicine) and as the plunger is more towards the front of the syringe body, at some stage, the collar on the plunger will pass underneath the engagement part of the syringe body and there may be slight deflection to enable this to happen (the tapering edge on the collar facilitating this action) but once the plunger has moved further forwardly, subsequent retraction of the plunger is prevented by engagement of the collar against the engagement part of the syringe body.

The syringe will typically be provided with a thumb pad. The thumb pad may have different shapes and configurations and may be substantially flat, or domed on the outside or something else. The thumb pad may be solid or hollow and may be formed integrally with the plunger or formed separately and attached to the plunger. The thumb pad contains a seal that seals against the open rear end portion of the syringe body. The seal may be provided along a bottom surface of the thumb pad such that the thumb pad seals against the outer most peripheral edge of the syringe body as opposed to an inside wall of the syringe body. The seal may be provided at a peripheral edge of the thumb pad and it is preferred that the peripheral edge is tapered thereby making it difficult to try to prise the thumb pad back into the retracted position.

It is preferred that the plunger lock mechanism is designed and positioned such that when the plunger lock mechanism is engaged (see for instance FIG. 12), this also presses the seal on the thumb pad against the outer most peripheral edge of the syringe body. Thus, there is a significant advantage in having the thumb pad and particularly the seal on the thumb pad positioned such that the thumb pad overlies the outer most edge of the syringe body as this can provide a good seal, as opposed to sealing against the inside edge of the syringe body, which can still leak particularly if the relatively thin wall body is squeezed and slightly deformed out of shape.

It will be clearly understood that, if a prior art publication is referred to herein, this reference does not constitute an admission that the publication forms part of the common general knowledge in the art in Australia or in any other country.

### BRIEF DESCRIPTION OF THE DRAWINGS

Embodiments of the invention will be described with reference to the following drawings in which:

FIG. 1. Illustrates in cross-section a front portion of the syringe with the plunger moving towards the forward part of the syringe but not yet contacting any part of the shoot back mechanism.

FIG. 2. Illustrates in cross-section the plunger just beginning to make contact with the raised portion of the outer part of the needle holder, and also just beginning to make contact with the “arrowhead” configuration on the inner part of the needle holder. At this stage, there is still an appreciable “dead space”, and the shoot back mechanism has not yet been triggered.

FIG. 3. Illustrates in cross-section the plunger having been pushed forwardly a little further than the position of FIG. 2, and illustrating the “arrowhead” configuration beginning to stretch the central part of the plunger seal, and also illustrating the plunger making greater contact with the outer part of the needle holder and having part of the plunger seal compressed to reduce the dead space. This position is just prior to triggering the shoot back mechanism.

FIG. 4. Illustrates the syringe after the shoot back mechanism has been triggered with the contaminated needle (and the inner part of the needle holder) being safely within the confines of the plunger.

FIG. 5. Illustrates a second embodiment of the invention which is to the design of the outer part of the needle holder to have a “chamfer” to further minimise dead space.

FIG. 6. Illustrates a third embodiment of the invention which is a syringe containing a pair of spaced apart visual indicators and the plunger in the substantially retracted position.

FIG. 7. Illustrates the plunger adjacent one of the visual indicators and just prior to triggering the retraction mechanism.

FIG. 8. Illustrates the plunger in the plunger locked position and where the plunger seal is positioned between the spaced apart visual indicator.

FIG. 9-FIG. 12 illustrate the main embodiment of the invention which are the features of the syringe which enables the syringe to be a “no-leak” syringe after retraction thereby enabling the syringe to be used for radioactive medicines or other hazardous materials, and in particular:

FIG. 9. Illustrates the syringe during retraction of the needle.

FIG. 10. Illustrates the syringe after full retraction.

FIG. 11. Is a close up of the front seal on the piston after retraction and showing the self-healing properties.

FIG. 12. Is a close up of the rear portion of the syringe showing the seal on the thumb pad.

### DETAILED DESCRIPTION OF THE INVENTION

Referring to the figures, the figures illustrate a preferred embodiments of a single use syringe having a retraction mechanism which comprises a “shoot back” mechanism. The mechanism according to the preferred embodiments may comprise the ones described in our earlier patent applications PCT/AU01/00183 and PCT/AU2006/00462 which are incorporated herein by cross reference.

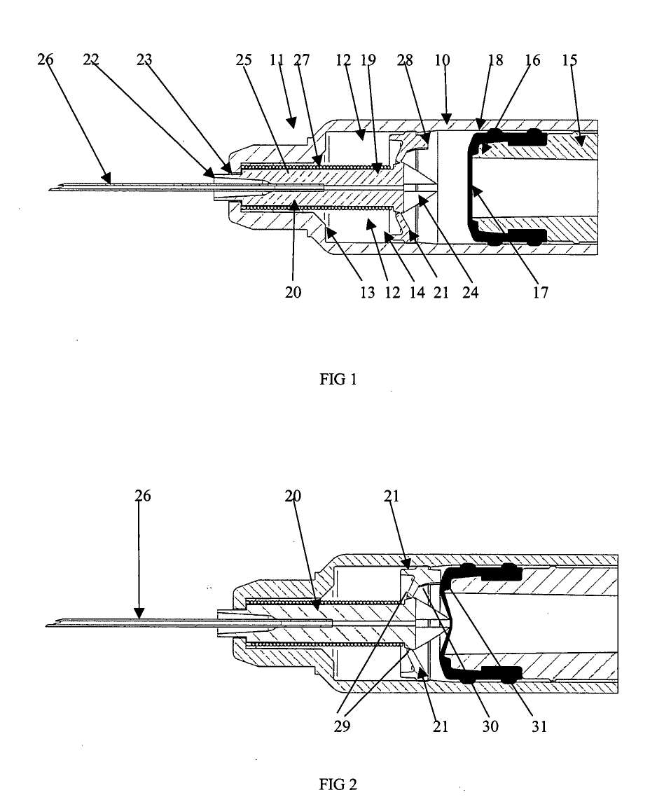

Various types of plunger locking mechanisms are envisaged, but for the purposes of illustration in a preferred embodiment, the following plunger lock may be used, and as illustrated in FIGS. 1-4.

Referring to the drawings and initially to FIG. 1, there is illustrated in cross-section the front part of a single use syringe. Briefly, the syringe comprises the following components: a syringe body 10 which has a front end area 11 which contains a longitudinal passageway 12, the passageway having a front portion 13 and a rear portion 14, a plunger 15 which is hollow and which contains a front face 16, a pierceable seal 17 which extends over the front face 16 of plunger 15, and which has a thickened edge portion 18, a needle holder 19 which comprises an inner part 20 and an outer part 21, the inner part further having a front portion 22 that extends through an opening 23 in the front of syringe body 11, a rear portion 24 which has an arrowhead type configuration, and an intermediate body portion 25, a needle (puncture needle) 26 that is fixed to the inner part 20, a spring 27 which extends about the intermediate body portion 25. Further more particular details will be described below.

The syringe body 10 has a front end area 11 which has a particular profile (see FIG. 1). The particular profile contains a forward most opening 23, a narrower diameter passageway behind opening 23 and to contain part of the intermediate body portion 25, and a large diameter passageway 12 which essentially forms an extension of the internal bore of the syringe body through which the plunger 15 can pass.

The inner part 20 of the needle holder 19 cannot move through opening 23 (by virtue of intermediate body portion 25 having a larger diameter than the front portion 22 and this diameter being larger than opening 23). Thus, if the plunger 15 pushes against the inner part 20, inner part 20 cannot move.

One end of spring 27 abuts against the end of the narrower diameter passageway behind opening 23, and the other end of spring 27 abuts against a small shoulder just behind the arrowhead configuration of the rear portion 24 of inner part 20. With this configuration, the spring is compressed and biases the inner part 20 into the shoot back position which is inside the hollow interior of plunger 15. However, this is prevented by the inner part 20 being attached to the outer part 21.

Outer part 21 in the particular embodiment is annular in configuration and extends entirely about inner part 20 and in the area of the arrowhead configuration of the inner part, this being illustrated in FIG. 1. Outer part 21 has an outer face 28 which presses against the inside wall of passageway 12. A small abutment in the inside wall may be provided to positively position the outer part 21 in place, and to prevent the outer part from moving towards the plunger. The outer part is attached to the inner part by a frangible portion 29 which is referenced in FIG. 2. The frangible portion 29 in the particular embodiment extends entirely between the inner part and the outer part. This prevents fluid from leaking part the needle holder and into a forward part of passageway 12.

The outer part 21 contains a raised portion 30 (best illustrated in FIG. 2) but also illustrated in the other figures) which extends only partway (typically about quarter) about the outer part. The raised portion projects in the direction of plunger 15 and functions to reduce the pressure required to trigger the shoot back mechanism as will be described in greater detail below.

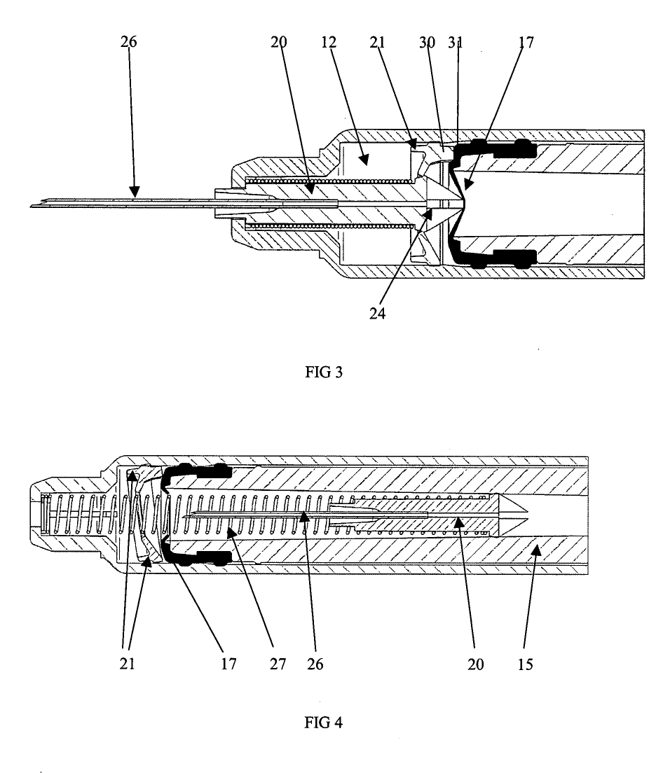

The triggering of the shoot back mechanism is somewhat similar to that described in our earlier international patent application given above, and basically occurs when plunger 15 is pressed hard up against the needle holder which is progressively illustrated in FIGS. 2-3. As this happens, the plunger contacts the outer part 21 and further forward pushing of the plunger enables the outer part to be pushed forwardly along passageway 12. As the inner part 20 of the needle holder cannot move in the same way, continued pressing on the plunger will cause the frangible portion 29 to be broken thereby releasing the inner part 20 from the outer part 21. As soon as this happens, spring 27 shoots the inner part 20 (containing the puncture needle 26) through the front of plunger 15 and into the hollow interior of the plunger. The final position is illustrated in FIG. 4.

However, in the present invention, a modification has been made to provide certain advantages. The modification includes providing the raised portion 30 on only a portion of the outer part 21. Thus, as plunger 15 moves forwardly from the position illustrated in FIG. 1 to the position illustrated in FIG. 2, an edge 31 of the plunger contacts the raised portion 30 (see FIG. 2). At this stage, the remainder of the plunger does not yet contact the remainder of the outer part 21; instead, only part (edge 31) of the plunger contacts only part (the raised portion 30) of the outer part. This concentrates the force of the plunger on to only part of the outer part 21 and enables the frangible portion in this area to be broken. It is found that once part of the frangible portion is broken, the remainder of the frangible portion can be broken with much less force.

However, there is a further modification which also reduces the dead space. The dead space is defined as the volume that cannot be expelled from the syringe due to the design of the internal components. Clearly, the amount of dead space should be reduced as much as possible.

In the present embodiment, once the plunger is in the position illustrated in FIG. 2, there is only slight contact between the edge 31 of the plunger and the raised portion 30 of the outer part. The arrowhead portion of the inner part has also contacted the seal 17 and is beginning to stretch the seal and weaken the seal. However, at this stage, the shoot back mechanism has not been triggered in that the frangible portion has not yet been stretched and broken.

Instead, the plunger is moved slightly more forwardly as indicated in FIG. 3. The forward movement need not be much (perhaps 1 mm), but is sufficient to bring the seal 17 into relatively close contact with the internal edges of the needle holder to reduce the amount of dead space. To accommodate the raised portion 30, the edge 31 of seal 17 is relatively thick (compared to the remainder of the front portion of the seal) and can be compressed or squashed. Therefore, in the position illustrated in FIG. 3, the plunger has been moved more forwardly and the raised portion 30 has been compressed into the rubbery edge 31 still without triggering the shoot back mechanism. It can be seen that in this particular position, the remainder of the front of the plunger is now in contact with the rest of the outer part 21. In this position, the amount of dead space has been reduced with respect to the amount of dead space when the plunger was in the position illustrated in FIG. 2.

Further forward movement of the plunger will now push the outer part 21 along passageway 12 and because of raised portion 30, the greatest force (and therefore greatest pushing action) will be on the raised portion 30 which will cause a preferential breaking or rupture of the frangible portion in this area only. This results in a reduced force being required to break the frangible portion. Once the frangible portion has been preferentially ruptured, it is found that less force is required to completely rupture the remainder of the frangible portion. As the plunger is pushed forwardly, the seal 17 is also stretched even further by the arrowhead configuration of the inner part 20 and the seal can be ruptured via the arrowhead configuration, or release of the spring is sufficient to now shoot the inner part and the contaminated needle through the seal and into the hollow plunger is illustrated in FIG. 4. The outer part 21 is slightly tilted as it is pushed forwardly.

The arrangement is found to reduce the force required to trigger the shoot back mechanism from approximately 9 kg to between 2-3 kg. The shoot back mechanism is still robust and has not been made “flimsy” in order to reduce the force required. This is achieved by having a raised portion on the outer part 21 and having the seal on the plunger being relatively conventional and not requiring any complicated design on the front of the plunger. By enabling the plunger to be relatively conventional, the raised portion 30 can be partially pushed into (compressed) into the edge of the seal to reduce the dead space prior to triggering the shoot back mechanism. It is not considered that such a relatively simple and reliable mechanism is possible with a complicated plunger shape.

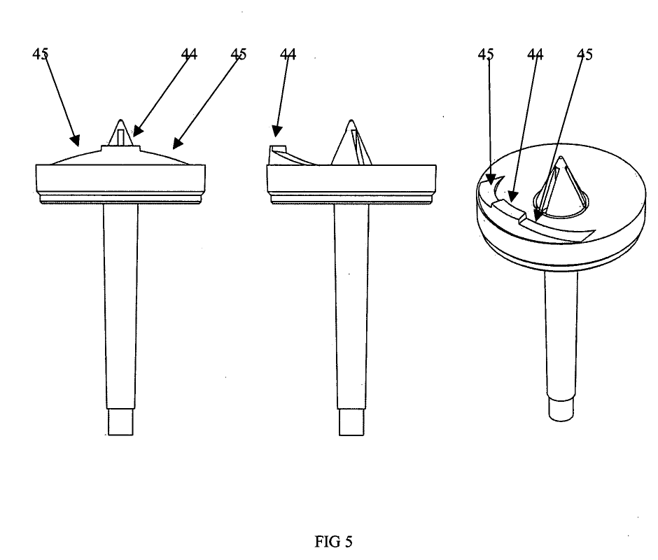

If desired, each side of abutment 44 may be provided with a “chamfer” 45 to further minimise dead space. A typical chamfer is illustrated in FIG. 5.

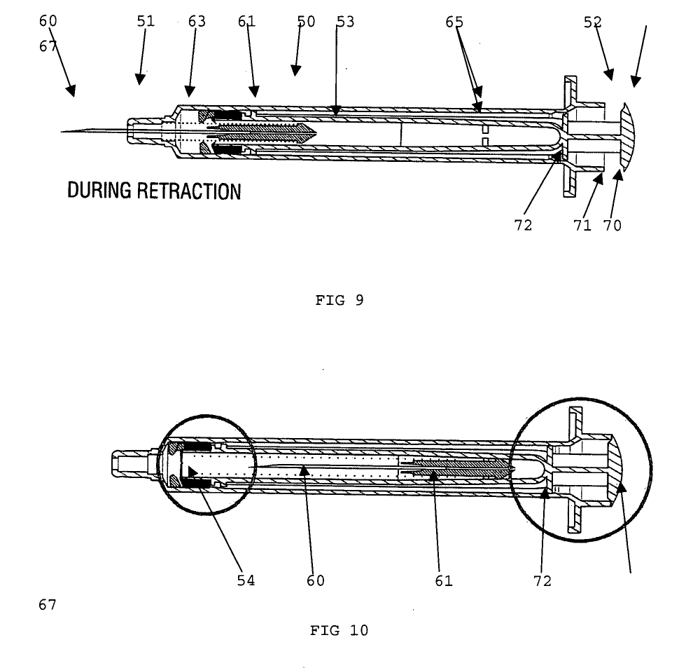

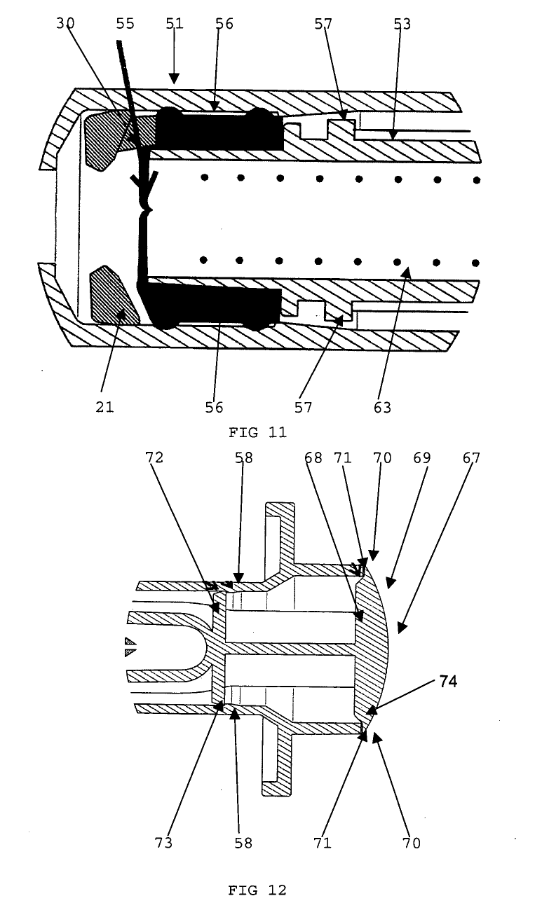

Reference will now be had to FIGS. 9-12 which illustrate the various parts of the syringe that allows the syringe to be used for nuclear medicine or other hazardous materials because when the needle is retracted into the syringe body, the syringe does not leak. FIG. 9 illustrates the syringe at the point where retraction of the needle is taking place and FIG. 10 illustrates the syringe where the needle has been retracted safely into the confines of the plunger body. The syringe has a syringe body 50 having a front portion 51 and an open rear end portion 52. A plunger 53 is provided which has an open front end 54 and a closed rear end to define an internal area into which the needle can be held after retraction. The open front end 54 is closed by a pierceable self-healing seal 55 which is best illustrated in FIG. 11. Seal 55 forms part of a larger seal 56 which extends about the front side wall of plunger 53 and larger seal 56 functions as the ordinary plunger seal that prevents liquid from flowing past the plunger. In this particular embodiment, seal 55 and seal 56 are formed integrally. Behind plunger seal 56 (see FIG. 11) is an annular rib 57. The function of rib 57 is to prevent the plunger from being pulled entirely out of the syringe body (e.g. to prevent over retraction) by contacting an engagement part 58 in the syringe body (illustrated in FIG. 12 and the engagement part 58 will be described in greater detail below). Briefly however, if the plunger is pulled too far back (e.g. drawing up a radioactive fluid from a vial), at some stage, rib 57 will strike engagement part 58 and this will prevent the plunger from being retracted entirely out of the syringe body which also makes the syringe safe for nuclear medicine etc. this part may be called an “over retraction protection lock”.

The syringe contains a needle 60 attached to a needle holder, and the needle holder contains an inner part 61 to which the needle is directly attached, and an outer part 62, the inner part 61 and the outer part 62 being connected by a breakable or frangible member. A spring 63 is also provided, and these features are identical or similar to that described with reference to FIGS. 1-4.

Thus, when the plunger is pushed to the front of the syringe body, at some stage, the plunger will trigger the retraction mechanism and the inner part 61+needle 60+spring 63 will shoot through the self-healing seal 55 and into the safety of the plunger body (see FIG. 10). Seal 55 will then close due to its resilient properties. At this stage, any hazardous material remaining in the needle or on the outside of the needle cannot pass through seal 55 and back out of the open front of the syringe body.

The rear of plunger 53 is formed with a number of small rectangular openings 65 (see FIG. 9) which pass through the wall of plunger 53. These openings 65 function as a catch for inner part 61. Referring to FIG. 10, the inner part 61 (and particularly the “arrowhead” portion 66 of inner part 61) shoots past opening 65 when the needle is retracted and any attempt to fiddle the needle back out of the plunger is prevented by the arrowhead portion 66 locking into the openings 65. However, this also results in an opening being formed through the plunger wall and therefore enabling hazardous materials to pass from the inside of the plunger and into the inside of the syringe body by leaking through the opening 65.

For this reason, the plunger 53 is formed with a specially designed thumb pad 67 and this is perhaps best illustrated in FIG. 12. Thumb pad 67 contains a flat inner face 68 and a domed outer face 69. The inner face of the thumb pad 67 is provided with an annular seal 70. The design of the thumb pad 67 is such that the seal 70 abuts against and seals against the outermost open edge 71 on the back of the syringe body (See also FIG. 9). Thus, when the thumb pad is in the sealed position illustrated in FIG. 10 and FIG. 12, any liquid that may pass between the plunger and the syringe body cannot flow out of the rear of the syringe. Therefore, in the position illustrated in FIG. 10, the syringe is completely closed and leak proof and it is virtually impossible for any potentially hazardous liquid to leak either out of the front of the syringe (because of seal 55 and seal 56) or out of the rear of the syringe (because of seal 70).

A plunger lock mechanism is provided to lock the plunger against retraction when the plunger is in the fully forward position (this is where the plunger has triggered the shoot back mechanism and the needle has been retracted within the plunger body). This mechanism is required because otherwise the integrity of seal 70 may be compromised because it might be simple to simply retract the plunger slightly to release the sealing engagement between seal 70 and edge 71. The plunger lock mechanism is best illustrated in FIG. 12 and comprises a collar 72 which extends from a rear portion of the plunger 53. Collar 72 has a tapered edge 73. This enables the plunger to be initially pulled back (to draw fluid into the syringe) and then be pushed forwardly where, at some stage, collar 72 will ride underneath the engagement part 58. However, once collar 72 is in front of engagement part 58 (see FIG. 12), the collar will lock against engagement part 58 to prevent retraction of the plunger. The design and positioning of the collar is such that when the collar engages against engagement part 58, it also ensures that the seal 70 on the thumb pad 67 sealingly engages against edge 71 on the back of the syringe body.

The engagement part 58 comprises a slightly thickened wall portion of the syringe body to form an abutment/shoulder etc against which the collar 72 can lock. It is also envisaged that some additional sealing may be provided in this area.

The plunger lock mechanism (e.g. collar 72) keeps the plunger in the field position and ensures that it is difficult if not impossible to attempt to retract the piston.

Thumb pad 67 comprises a tapered portion 74 (see FIG. 12) to allow or to guide thumb pad 67 into the sealing engagement illustrated in FIG. 12.

If, during the triggering process, some of the spring 63 remains protruding through seal 55, it is found that seal 55 will still feel itself about the spring thereby preventing any fluid from leaking through the seal.

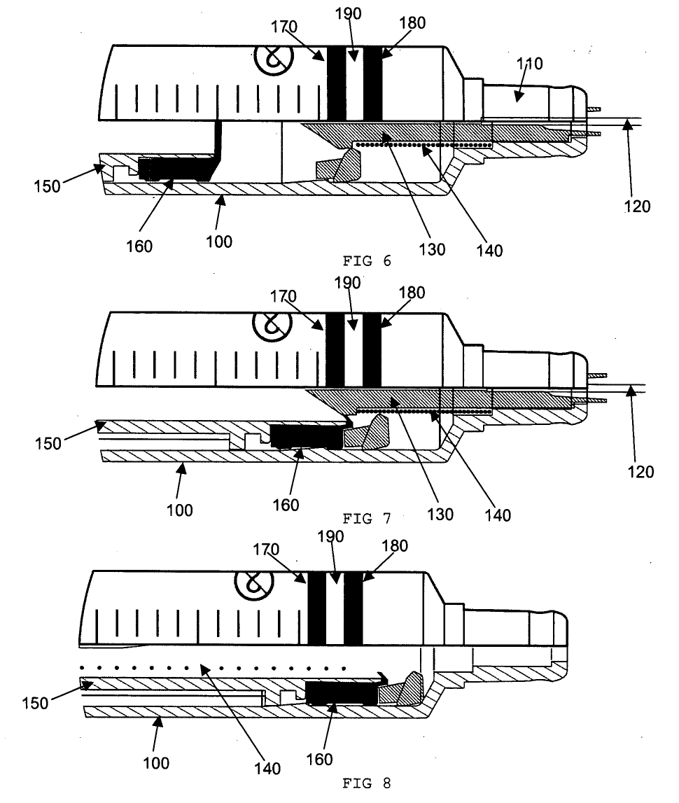

Referring to FIG. 6, there is illustrated the front area of a syringe which comprises a syringe barrel 100, a front portion 110 on the front of the syringe barrel, a needle 120, and a needle holder 130 which is positioned inside the front part of the syringe barrel and a compressed spring 140 which is positioned about the needle holder and more detail of which can be found below and in the above patent applications.

A plunger 150 contains a front seal 160 which is usually black or a contrasting colour.

The needle holder 130 comprises an outer portion which is held by the inside wall of the barrel and an inner portion which contains the needle and the spring. As the plunger is pushed forwardly, the front seal 160 presses against the outer portion and dislodges the outer portion relative to the inner portion. This can be seen as the “triggering” position and as soon as this occurs, the spring can expand and will shoot the inner portion+spring+needle into the hollow plunger body (or the barrel).

A coloured band 170 is positioned about the barrel and is positioned to extend about the outer portion of the needle holder. Thus, as the plunger is pushed forwardly to the position illustrated in FIG. 7, the plunger seal 160 is adjacent band 170 and there is a clear visual indication that the triggering action is about to occur and that all the medicine has been expelled from the syringe.

Further forward movement of plunger 150 will trigger the retraction mechanism and will cause the needle to shoot back into the confines of the plunger body (see FIG. 8).

As the plunger moves further forward, the plunger seal 160 will move past band 170 and towards a second band 180. Band 170 and band 180 are separated by a clear portion 190 of the barrel (usually the entire barrel is made of the same clear or substantially clear material). When the plunger seal 160 is positioned between band 170 and band 180 (see FIG. 8), a plunger locking mechanism engages to lock the plunger against retraction.

There is a clear visual indication that the plunger is locked by virtue of viewing the plunger seal in position between band 170 and band 180.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

1. A single use syringe having a needle which retracts into the syringe after use, and when the needle is retracted, the syringe is sealed to prevent any liquid still on or in the needle from leaking either from the front of the syringe or from the rear of the syringe, the syringe comprising:
a syringe body having a front portion and an open rear portion,
a plunger having a front and a rear and which is moveable in the syringe body from a retracted position where the front of the plunger is in the rear portion of the syringe body to a extended position where the plunger has moved in the syringe body towards the front of the syringe body,
a pierceable self healing seal extending over the front of the plunger and through which the needle can pierce when the needle is retracted, the seal being substantially closed after the needle has passed through the seal,
a seal on the side wall of the plunger to seal against the inner wall of the syringe body during movement of the plunger in the syringe body
a needle holder which is positioned in the front of the syringe body,
a needle attached to the needle holder,
a triggering mechanism to enable the needle to be retracted into the syringe body and/or the plunger body,
a plunger lock mechanism to lock the plunger against retraction when the needle has been retracted,
a thumb pad on the rear of the plunger, and
a seal on the thumb pad that seals against the open rear end portion of the syringe body to prevent liquid from leaking from the rear end portion of the syringe body when the needle has been retracted,
whereby, when the needle is retracted after use, the pierceable self healing seal extending over the front of the plunger and the seal on the side wall of the plunger prevents any leakage of liquid from the needle through the front of the syringe and the seal on the thumb pad prevents any leakage of liquid from the needle through the rear of the syringe.

2. The syringe as claimed in claim 1, wherein the plunger lock mechanism comprises a projection extending from the plunger and which engages with a projection extending inwardly from an internal wall of the syringe body to prevent the plunger from being retracted.

3. The syringe as claimed in claim 2, wherein the projection extending from the plunger comprises a circumferential disk extending from a rear portion of the plunger and the projection extending inwardly from the internal wall of the syringe body comprises a circumferential abutment positioned in the rear portion of the syringe body.

4. The syringe as claimed in claim 2, wherein the thumb pad comprises an inner face and an outer face, the seal on the thumb pad being provided about an edge of the inner face and adapted to seal against the open rear end portion of the syringe body.

5. The syringe as claimed in claim 4, wherein the plunger lock mechanism is positioned in the syringe such that when the plunger is locked to the syringe body by the plunger lock mechanism, the seal on the thumb pad is in sealing engagement with the open end rear portion of the syringe body.

6. The syringe as claimed in claim 5 wherein the triggering mechanism has been triggered to retract the needle when the plunger is locked to the syringe body against retraction.

7. The syringe as claimed in claim 5, wherein the inner face of the thumb pad has a tapered portion (74) extending immediately behind the seal.

8. The syringe as claimed in claim 1, wherein the front of the plunger contains a circumferential seal (56) which sealingly and slidingly engages the plunger to the inside wall of the syringe body during movement of the plunger in the syringe body, the circumferential seal being formed integrally with the pierceable self healing seal (55).

9. The syringe as claimed in claim 8, wherein the plunger has a hollow body into which the retracted needle and part of the needle holder can pass, the plunger having a side wall which contains at least one small opening (65) which functions as a catch to prevent the retracted needle from being removed from the plunger.

10. The syringe as claimed in claim 4, wherein the needle holder comprises an inner part to which the needle is attached, and an outer part which is formed integrally with the inner part, a frangible portion between the inner part and the outer part, the outer part being held in the front of the syringe body, and a spring which extends about the inner part of the needle holder.

11. The syringe as claimed in claim 10 wherein the inner wall of the syringe body comprises an abutment and the outer part is held in the front of the syringe body by engaging with the abutment.

12. The syringe as claimed in claim 11, wherein the triggering mechanism comprises a front portion of the plunger contacting the outer part of the needle holder and dislodging at least part of the outer part of the needle holder from the inner part along the frangible portion.

13. The syringe as claimed in claim 12, wherein the outer part of the needle holder comprises a bump which faces the plunger and initial contact is made by the plunger abutting against the bump to begin the triggering process with a reduced force.

14. The syringe as claimed in claim 10, comprising at least one visual indicator positioned at or adjacent the triggering position and a second visual indicator positioned forwardly of the first visual indicator, the syringe body being at least partially clear between the first the visual indicator and the second visual indicator such that the plunger seal can be viewed therebetween when the plunger is pushed into that position.

15. The syringe as claimed in claim 14, wherein each visual indicator comprises a coloured band extending about the syringe body.

16. A single use syringe comprising:
a syringe body having a front portion and an open rear portion,
a plunger having a front and a rear and which is moveable in the syringe body from a retracted position where the front of the plunger is in the rear portion of the syringe body to a extended position where the plunger has moved in the syringe body towards the front of the syringe body,
a pierceable self healing seal extending over the front of the plunger and through which the needle can pierce when the needle is retracted, the seal being substantially closed after the needle has passed through the seal,
a needle holder which is positioned in the front of the syringe body,
a needle attached to the needle holder,
a triggering mechanism to enable the needle to be retracted into the syringe body and/or the plunger body,
a plunger lock mechanism to lock the plunger against retraction when the needle has been retracted,
a thumb pad on the rear of the plunger, and
a seal on the thumb pad that seals against the open rear end portion of the syringe body to prevent liquid from leaking from the rear end portion of the syringe body when the needle has been retracted.
